(19) United States
(12) Patent Application Publication
(10) Pub. No.: US 2023/0372247 A1
(43) Pub. Date: Nov. 23, 2023

(54) SELF-DISINTEGRATING EXCIPIENT COMPOSITION FOR SOLID ORAL DOSAGE FORMS

(71) Applicant: Sigachi Industries Limited, Telangana (IN)

(72) Inventors: Monika TOMAR, Gujarat (IN); Amit Raj SINHA, Telangana (IN)

(73) Assignee: Sigachi Industries Limited, Telangana (IN)

(21) Appl. No.: 17/947,858

(22) Filed: Sep. 19, 2022

(30) Foreign Application Priority Data
May 17, 2022 (IN) 202241028363

(51) Int. Cl. A61K 9/20

(52) U.S. Cl. CPC A61K 9/2054; A61K 9/2004

(57) ABSTRACT

The present invention relates to a cellulose based self-disintegrating co-processed excipient composition comprising of a combination of cellulose binder, microcrystalline cellulose and cellulose super disintegrant, croscarmellose sodium. The present invention also relates to process of preparing the self-disintegrating excipient composition and its use in pharmaceutical and nutraceutical formulation.

### FIELD OF THE INVENTION

The present invention generally relates to excipient composition. Specifically, the present invention relates to a cellulose based self-disintegrating co-processed excipient composition comprising of a combination of cellulose binder and cellulose super disintegrant. The present invention also relates to process of preparing the self-disintegrating excipient composition and its use in pharmaceutical and nutraceutical formulation.

### BACKGROUND OF THE INVENTION

Tablets represent the preferred and most commonly dispensed pharmaceutical dosage form for administering active pharmaceutical ingredients (APIs). Wet granulation, Dry granulation, and Direct Compression are three methods that are widely used to manufacture tablets.

Wet granulation method is performed by binding the powder together by adhesion rather than by compaction. The tensile strength of bonding increases as the amount of liquid added is increased in the Wet granulation method.

Dry Granulation method is a powder agglomeration process used to improve the flowability of powders by increasing the particle size (granules). This operation is achieved using a roller press, which can have different configurations and equipment.

Direct compression is the most widely used and easiest manufacturing option, making it easiest to control and least expensive. Minimizing the cost of goods and improving manufacturing output efficiency has motivated companies to use direct compression as a preferred method of tablet manufacturing. The direct compression (DC) method is simple to manufacture tablet, wherein, first binder and disintegrant are blended followed by glidant and active ingredients are added and blended again. The blended ingredients are finally lubricated to give compressed tablets. In direct compression method, selection of excipient is crucial. Excipients dictate the success of direct compression, notably by optimizing powder formulation compatibility and flow.

Thus there has been a surge in creating excipients specifically designed to meet these needs for direct compression. Greater scientific understanding of tablet manufacturing coupled with effective application of the principles of material science and particle engineering has resulted in several improved direct compression excipients, individual excipient and co-processed excipient. Despite this, significant practical disadvantages of direct compression remain relative to granulation, and this is partly due to the limitations of direct compression excipients. For instance, in formulating high-dose APIs, a much higher level of excipient is required relative in wet or dry granulation method and hence, the tablets are much bigger. Creating excipients to enable direct compression of high-dose APIs requires the knowledge of the relationship between fundamental material properties and excipient functionalities.

Excipients play a very important role to make formulation. Excipient should have few properties such as, inactive with drugs, free flowing, and show chemical and physical stability. Excipient should have high bulk density and at least 100-micron particle size and loss on dying would be less than 7% for normal API's and less than 1.5% for moisture sensitive API's.

It is necessary to have excipients with excellent functional properties to compensate for the poor mechanical properties and low aqueous solubility of the emerging active ingredients. Therefore, around 80% of the current drugs are not suitable for direct compression and more advanced excipients are required. Further, conventional grades of excipients cannot accommodate the technologically advanced high speed rotary tablet presses which require a powder with excellent flow, good compressibility, compatibility, particle size distribution and homogeneity of the ingredients. Co-processed excipients have been created to enhance the functional properties of the excipients and reduce their drawbacks. Co-processing is defined as the combination of two or more excipients by a physical process, wet process. Co-processed excipients are adequate for direct compression since they become multifunctional and thus, their dilution potential is high, eliminating the need for many excipients in a formulation. In some cases, they are able to hold up to 50% of the drug in a formulation rendering compacts good tablets.

Tablets are formed by the application of pressure to the tablet formulation on a tablet press. A tablet press includes a lower punch which fits into a die from the bottom and an upper punch having a corresponding shape and dimension, which enters the die cavity from the top after the tablet for mutation fills the die cavity. The tablet is formed by pressure applied on the lower and upper punches. The ability of the tablet formulation to flow freely into the die is important in order to ensure that there is a uniform filling of the die and a continuous movement of the tablet formulation from its source. The tablet must also eject cleanly from the die following compression. Typically, a lubricant is added to the tablet formulation to avoid sticking to the die and to cause the formulation to flow freely. Because of its inherent compactability characteristics, microcrystalline cellulose is widely used as an excipient in tablet formulations. Good binding and disintegration properties are obtained with microcrystalline cellulose when used it is used in direct compression tablet formulations. However, microcrystalline cellulose can have lubricant sensitivity. Lubricant sensitivity refers to the reduction in bonding between the plastically deforming particles in the powder due to the addition of lubricant, which leads to reduction in tablet strength or hardness. Lubricant sensitivity is the ratio of the unlubricated compactability of the tablet formulation to the lubricated compactability of the tablet formulation. Tablet manufacturing has changed by the introduction of the direct compression process and high-speed machines. These two developments have increased the demands on the functionality of excipient in terms of flow and compression properties.

Co-processing is based on the novel concept of two or more excipients interacting at the sub particle level, the objective of which is to provide a synergy of functionality improvements as well as masking the undesirable properties of individual excipients. The availability of a large number of the excipients for co-processing ensures numerous possibilities to produce tailor-made “designer excipients” to address specific functionality requirements. Co-processed excipients are prepared by incorporating one excipient into the particle structure of another excipient using processes such as co-drying. Thus, they are simple physical mixtures of two or more existing excipients mixed at the particle level. Development of co-processed excipients starts with the selection of the excipients to be combined, their targeted proportion, selection of preparation method to get optimized product with desired physio-chemical parameters and it ends with minimizing avoidance with batch-to-batch variations. An excipient of reasonable price must be combined with the optimal amount of a functional material in order to obtain integrated product, with superior functionality than the simple mixture of components. Co-processing is interesting because the products are physically modified in a special way without altering the chemical structure. A fixed and homogenous distribution for the components is achieved by embedding them into mini granules.

Thus, a need exists for an co-processed excipient with superior functionalities particularly high compactability, low lubricant sensitivity, and low ejection force profile that makes it an ideal for tablet formulations, particularly for direct compression.

### OBJECT OF THE INVENTION

An object of the present invention is to provide a self-disintegrating co-processed excipient composition comprising of a combination of binder and super disintegrant.

Yet another object of the invention is to provide a self-disintegrating co-processed excipient composition comprising of cellulose binder and cellulose super disintegrant having excellent flow, and good compressibility tablet profile.

Yet another object of the present invention is to method of preparing the self-disintegrating co-processed excipient composition comprising of cellulose binder and cellulose super disintegrant.

### SUMMARY OF THE INVENTION

The present invention relates to a cellulose based self-disintegrating co-processed excipient composition comprising of a combination of cellulose binder and cellulose super disintegrant. The present invention also relates to a process of preparing the self-disintegrating excipient composition and its use in pharmaceutical and nutraceutical formulation.

In one aspect, the present invention relates to a self-disintegrating co-processed excipient composition comprising of:
(a) binder represents microcrystalline cellulose; and
(b) disintegrant represents croscarmellose sodium; wherein the ratio of binder to disintegrant is in a range of 99.5:0.5 to 85:15.

In another aspect of the present invention, the co-processed excipient composition further comprises lubricating agents.

In another aspect of the present invention, the lubricating agent is selected from magnesium stearate, sodium stearyl fumarate, stearic acid, and talc.

In another aspect of the present invention, the lubricating agent is present in an amount ranges from 0.2% to 1% by weight of the composition.

In another aspect of the present invention, the co-processed excipient composition having a weight loss on drying of no more than about 7% by weight.

In another aspect of the present invention, the particle size distribution of microcrystalline cellulose and croscarmellose sodium is in a range of 30 microns to 200 microns.

In another aspect of the present invention, the composition having a bulk density in a range of 0.20 g/ml to 0.50 g/ml.

In another aspect of the present invention, pH of the composition is in a range of 5.0 to 7.5.

In another aspect, the present invention relates to a method of preparation of a self-disintegrating co-processed excipient composition comprising the steps of:
(a) mixing a binder, a disintegrant individually with water to obtain an individual homogenous slurry;
(b) mixing the individual homogenous slurry of binder and disintegrant under stirring at a temperature in a range of 30° C. to 100° C. to obtain a homogenous mixture slurry;
(c) spray or flash drying the homogenous mixture slurry to obtain the co-processed excipient composition.

In another aspect of the present invention, the binder is microcrystalline cellulose; and the disintegrant is croscarmellose sodium.

In another aspect of the present invention, the method of preparing a self-disintegrating co-processed excipient composition comprises the step of lubricating the homogenous mixture slurry.

Various objects, features, aspects and advantages of the inventive subject matter will become more apparent from the following detailed description of preferred embodiments.

### BRIEF DESCRIPTION OF THE DRAWINGS

FIG. 1A depicts the flow diagram of method of preparation of a self-disintegrating co-processed excipient composition by making individual slurry of MCC wet cake and CCS using demineralised water.

FIG. 1B depicts the flow diagram of method of preparation of a self-disintegrating co-processed excipient composition by adding individual slurry of MCC wet cake and CCS powder to demineralised water.

FIG. 1C depicts the flow diagram of method of preparation of a self-disintegrating co-processed excipient composition by adding MCC powder and CCS powder to demineralised water.

FIG. 2 shows the tablet hardness and compaction compression of self-disintegrating co-processed excipient composition of present invention and physical blend.

FIG. 3 shows the tablet hardness comparison of self-disintegrating co-processed excipient composition of present invention and physical blend with different percentage of croscarmellose sodium.

FIG. 4 shows the tablet disintegration time comparison of self-disintegrating co-processed excipient composition of present invention and physical blend with different percentage of croscarmellose sodium.

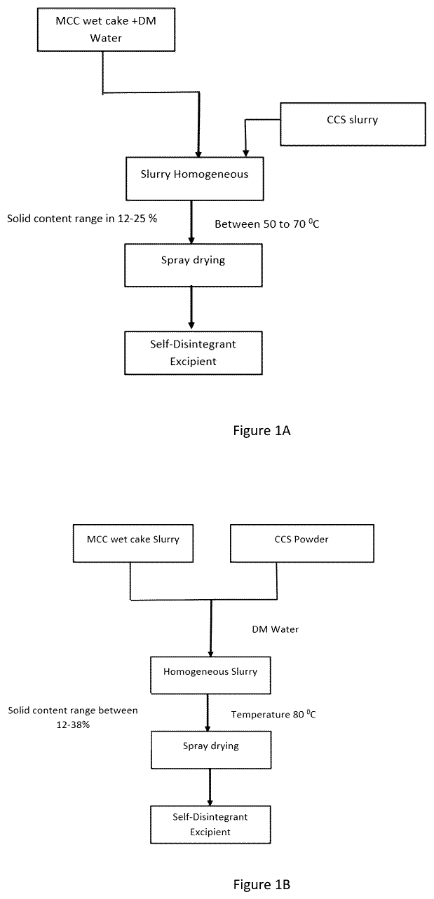

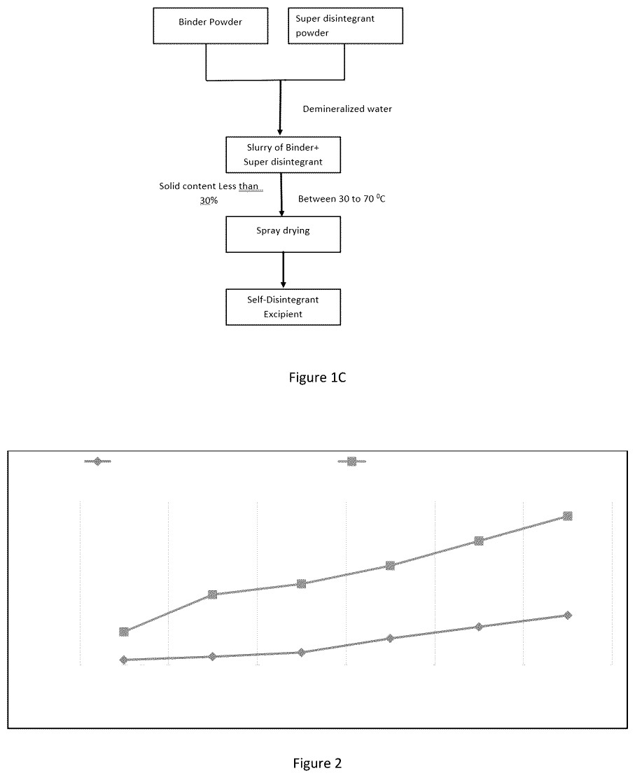

### DETAILED DESCRIPTION OF THE INVENTION:

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written

The description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In an embodiment, the present invention generally relates to excipient composition. Specifically, the present invention relates to a cellulose based self-disintegrating co-processed excipient composition comprising of a combination of cellulose binder and cellulose super disintegrant having excellent flow, and good compressibility tablet profile.

In an embodiment, the present invention relates to a self-disintegrating co-processed excipient composition comprising of:
(a) binder represents microcrystalline cellulose; and
(b) disintegrant represents croscarmellose sodium; wherein the ratio of binder to disintegrant is in a range of 99.5:0.5 to 85:15.

In an embodiment of the present invention, the microcrystalline cellulose can be HiCel™ SCG 90 M, HiCel™ LP200, HiCel™ 12, HiCel™ HD 90 M, HiCel™ 90 M, HiCel™ 50 M, HiCel™ LD 50 M and HiCel™ 25 M are use with normal API's to make tablet direct compression, dry granulation and wet granulation also. The HiCel™ XLM grades and HiCel™ 14 are used with moisture sensitive API's for making tablets.

In an embodiment of the present invention, the microcrystalline cellulose is present in an amount range of 99.5% to 85% by weight of the composition.

In another embodiment of the present invention, the microcrystalline cellulose is present in an amount 99.5%, 99%, 98.5%, 98%, 97.5%, 97%, 96.5%. 96%, 95.5%, 95%, 94.5%, 94%, 93.5%. 93%, 92.5%, 92%, 91.5%, 91, 90.5%, 90%, 89.5%, 89%, 88.5%, 88%, 87.5%, 87%, 86.5%, 86%, 85.5% and 85% by weight of the composition.

In an embodiment of the present invention, the croscarmellose sodium is present in an amount 0.5% to 15% by weight of the composition.

In another embodiment of the present invention, the croscarmellose sodium is present in an amount 0.5%, 1%, 1.5%, 2%, 2.5%, 3%, 3.5%, 4%, 4.5%. 5%, 5.5%, 6%, 6.5%, 7%, 7.5%. 8%, 8.5%, 9%, 9.5%, 10, 10.5%, 11%, 11.5%, 12%, 12.5%, 13%, 13.5%, 14%, 14.5%, and 15% by weight of the composition.

In an embodiment of the present invention, the self-disintegrating co-processed excipient composition includes lubricant.

In another embodiment of the present invention, the lubricant is selected from magnesium stearate, sodium stearyl fumarate, steric acid and Talc. Lubricant can be used in an amount ranges from 0.2% to 1% to make good quality of tablet.

In another embodiment of the present invention, the lubricant is present in an amount 0.2% to 1% by weight of the composition. Preferably, in an amount ranges from 0.5% to 1%.

In another embodiment of the present invention, the mean particle size distribution of the composition is in the range of 30 microns to 200 microns. Preferably in the range of 90 to 150 microns. More preferably in the range of 95 to 120 microns.

In another embodiment of the present invention, the bulk density of the composition is in a range of 0.20 g/ml to 0.50 g/ml. Preferably, the bulk density is in the range of 0.25 g/ml to 0.50 g/ml.

In yet another embodiment, the present invention relates to a manufacturing of the co-processed self-disintegrating excipients comprising the steps of:
(a) mixing a binder, a disintegrant individually with water to obtain an individual homogenous slurry;
(b) mixing the individual homogenous slurry of binder and disintegrant under stirring at a temperature in a range of 30° C. to 100° C. to obtain a homogenous mixture slurry; and
(c) spray or flash drying the homogenous mixture slurry to obtain the co-processed excipient composition.

In another embodiment of the present invention, the method of preparing a self-disintegrating co-processed excipient composition comprises the step of lubricating the homogenous mixture slurry.

In another embodiment of the present invention, the homogenous mixture slurry can be obtained by adding the slurry binder with water and powder disintegrant to water under stirring.

In another embodiment of the present invention, the homogenous mixture slurry can be obtained by adding powder binder and powder disintegrant to water under stirring.

In another embodiment of the present invention, solid content of the homogenous mixture slurry is about less than 38%. Preferably, solid content of the homogenous mixture slurry is about 12-38% .

In another embodiment of the present invention, temperature to obtain the homogenous mixture slurry is in the range of 30° C. to 100° C. Preferably in the temperature range of 30° C. to 80° C.

Accordingly to the present invention, the particulate product of the present invention can also be used as a component of a tablet, which may be prepared directly from a physical blend of the particulate product along with one or more additional ingredients such as an API or from a granulate prepared as described above (also possibly in combination with one or more additional ingredients).

In another embodiment of the present invention, moisture level of the composition of present invention is not more than about 7%.

In another embodiment of the present invention, the particulate product of the present invention is particularly useful as an excipient or binder-disintegrant in processes involving roller compaction, granulation, and/or tableting.

In another embodiment of the present invention, the co-processed self-disintegration composition holds desirable performance attributes that are not shown with corresponding physical blends of Microcrystalline Cellulose and Croscarmellose Sodium, in any ratio what so ever.

Tabletting is well known to those skilled in the art of tablet formation. A tablet press consists of a lower punch that fits into a die from the bottom, and an upper punch with a corresponding shape and dimension that enters the cavity of the die from the top after the tablet formulation has filled it.

In embodiment of the present invention, the method of preparation involves uniform filling of the die with continuous flow of dose formulation, the tablet formulation must be able to flow freely into the die.

In embodiment of the prevent invention, the method of preparation involves includes addition of lubricant to eliminate physical defects such as lamination, capping, double impression weight variations, facilitate expulsion of the tablet from the die following compaction, to prevent stickiness to the punch faces, and to facilitate extraction of the tablet from the die following compaction.

In another embodiment of the present invention, the following examples are presented for illustration only, and are not intended to limit the scope of the invention or appended claims

### EXAMPLE 1

### Composition:

| Ingredients | Weight | Weight % |
|---|---|---|
| MCC | 99.5 g | 99.5% |
| CCS | 0.5 g | 0.5% |

### Manufacturing Step:

### Step-1:

1. Weighed accurately binder, microcrystalline cellulose and super disintegrant, croscarmellose sodium.
2. Individual slurry of binder MCC and super disintegrant Croscarmellose sodium was made.

### Step-2:

1. Mixed both slurries together to obtain homogeneous slurry of MCC and CCS.
2. Adjusted solid content of the homogeneous slurry between 12% to 38% and on temperature 30° C. to 80° C. to obtain self-disintegrating slurry.

### Step-3:

The self-disintegrating slurry was dried with the help of bulk drier and spray drier to obtain self-disintegrating excipient composition.

### Results:

| Parameters | Co-process | Physical blend |
|---|---|---|
| Bulk density(g/CC) | 0.25 | 0.24 |
| Mean Particle (μm) | 99 | 98 |
| Loss on drying (%) | 3.8 | 5.0 |
| Hardness (N) | 185 | 80 |
| Disintegration time (Sec) | 100 | 180 |

The following compositions are prepared by following the manufacturing step of Example 1 with non-critical variations.

| Example | MCC | CCS |
|---|---|---|
| Example 2 | 99% | 1% |
| Example 3 | 98% | 2% |
| Example 4 | 98% | 2% |
| Example 5 | 94% | 6% |
| Example 6 | 92% | 8% |
| Example 7 | 90% | 10% |
| Example 8 | 88% | 12% |
| Example 9 | 86% | 14% |
| Example 10 | 85% | 15% |

### Results

| | Example 2 | | Example 3 | | Example 4 | |
|---|---|---|---|---|---|---|
| | Co- | Physical | Co- | Physical | Co- | Physical |
| Parameters | process | blend | process | blend | process | blend |
| Bulk density (g/CC) | 0.25 | 0.24 | 0.30 | 0.25 | 0.32 | 0.29 |
| Mean Particle | 99 | 98 | 104 | 98 | 120 | 92 |
| (μm) | | | | | | |
| Loss on drying (%) | 3.8 | 5.0 | 5.2 | 5.3 | 4.2 | 5.6 |
| Hardness (N) | 185 | 80 | 160 | 68 | 140 | 52 |
| Disintegration time | 100 | 180 | 80 | 160 | 66 | 154 |
| (Sec) | | | | | | |

| | Example 5 | | Example 6 | | Example 7 | |
|---|---|---|---|---|---|---|
| | Co- | Physical | Co- | Physical | Co- | Physical |
| Parameters | process | blend | process | blend | process | blend |
| Bulk density (g/CC) | 0.35 | 0.30 | 0.38 | 0.33 | 0.42 | 0.35 |
| Mean Particle | 115 | 89 | 98 | 79 | 99 | 73 |
| (μm) | | | | | | |
| Loss on drying (%) | 4.5 | 5.7 | 4.5 | 5.8 | 4.7 | 5.9 |
| Hardness (N) | 122 | 49 | 90 | 30 | 70 | 25 |
| Disintegration time | 49 | 133 | 33 | 120 | 28 | 80 |
| (Sec) | | | | | | |

| | Example 8 | | Example 9 | | Example 10 | |
|---|---|---|---|---|---|---|
| | Co- | Physical | Co- | Physical | Co- | Physical |
| Parameters | process | blend | process | blend | process | blend |
| Bulk density (g/CC) | 0.46 | 0.39 | 0.48 | 0.40 | 0.50 | 0.45 |
| Mean Particle | 99 | 74 | 98 | 75 | 98 | 78 |
| (μm) | | | | | | |
| Loss on drying (%) | 4.8 | 6.0 | 5.5 | 6.3 | 6.2 | 6.7 |
| Hardness (N) | 50 | 15 | 35 | 10 | 28 | 8.0 |
| Disintegration time | 60 | 16 | 8.0 | 50 | 4.0 | 43 |
| (Sec) | | | | | | |

The tablet hardness study of the composition of the present invention was carried out to assess the hardness properties of the composition. FIG. 2 demonstrates that tablet hardness and compaction compression of co-processed and physical blend. FIG. 2 show that the co-processed self-disintegrating excipient compositions of the present invention indicate greater tablet hardness than physical blend keeping all other parameters constant. As compression pressure increases, tablet hardness also increases, however the difference between tablet hardness obtained by co-processed self-disintegrated excipient and physical blend continues to increase. The results clearly show the non-linear increase in tablet hardness by usage of co-processed self-disintegrated excipient on increased compression force.

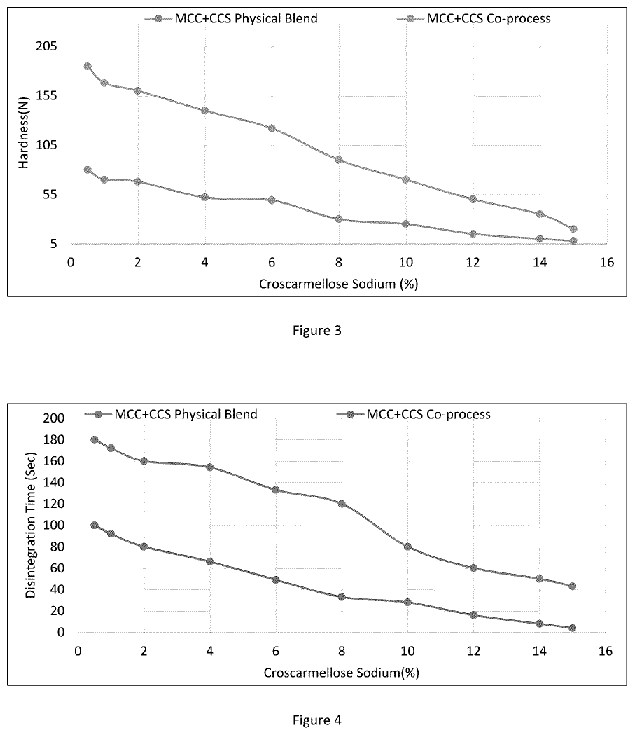

The tablet hardness study of the composition of the present invention with different percentage was studied. FIG. 3 represents tablet hardness comparison of co-processed self-disintegrated excipient composition of present invention and physical blend with different percentage of croscarmellose sodium (CCS). With the CCS percentage increasing, hardness of tablet shows a decreasing trend. It may be noted that the tablet hardness containing co-processed self-disintegrated excipient is always higher than those tablets made out of more than physical blend.

The disintegration time of the co-processed self-disintegrated excipient was studied. The FIG. 4 shows comparable tablet disintegration times of physical blend of MCC and CCS powder and a co-processed self-disintegrated excipient of the present invention. The results suggest that when CCS percent increased, the disintegration time decreased for each sample, although the excipient processed by co-processing performed better than the physical blend with lower disintegration time.

A skilled artisan will appreciate that the quantity and type of each ingredient can be used in different combinations or singly. All such variations and combinations would be falling within the scope of present disclosure

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

We claim:

1. A self-disintegrating co-processed excipient composition comprising of:
(a) binder represents microcrystalline cellulose; and
(b) disintegrant represents croscarmellose sodium;
wherein the ratio of binder to disintegrant is in a range of 99.5:0.5 to 85:15.

2. The self-disintegrating co-processed excipient composition as claimed in claim 1, further optionally comprises lubricating agents.

3. The self-disintegrating co-processed excipient composition as claimed in claim 2, wherein the lubricating agent is selected from stearate, sodium stearyl fumarate, stearic acid and talc

4. The self-disintegrating co-processed excipient composition as claimed in claim 2 or 3, wherein the lubricating agent is present in an amount ranges from 0.2% to 1% by weight of the composition.

5. The self-disintegrating co-processed excipient composition as claimed in claim 1, wherein the co-processed excipient composition having a weight loss on drying of no more than about 7% by weight.

6. The self-disintegrating co-processed excipient composition as claimed in claim 1, wherein particle size distribution of the microcrystalline cellulose and croscarmellose sodium is in a range of 30 microns to 200 microns.

7. The self-disintegrating co-processed excipient composition as claimed in claim 1, wherein the composition having a bulk density in a range of 0.20 g/ml to 0.50 g/ml.

8. The self-disintegrating co-processed excipient composition as claimed in claim 1, wherein pH of the composition is in a range of 5.0 to 7.5.

9. A method of preparation of a self-disintegrating co-processed excipient composition comprising the steps of:
(d) mixing a binder, a disintegrant individually with water to obtain an individual homogenous slurry;
(e) mixing the individual homogenous slurry of binder and disintegrant under stirring at a temperature in a range of 30° C. to 100° C. to obtain a homogenous mixture slurry; and
(f) spray or flash drying the homogenous mixture slurry to obtain the co-processed excipient composition.

10. The method of preparing a self-disintegrating co-processed excipient composition as claimed in claim 9, further optionally comprises the step of lubricating the homogenous mixture slurry.
